(19) United States
(12) Patent Application Publication
(10) Pub. No.: US 2002/0072085 A1
(43) Pub. Date: Jun. 13, 2002

(54) Method and device for fluid quality measurement

(76) Inventors: Michael S. Brunner, Roswell, GA (US); Ning Wei, Roswell, GA (US); David W. Koenig, Menasha, WI (US)

Correspondence Address: Neil C. Jones, Nelson Mullins Riley & Scarborough, Keenan Building, Third Floor, 1330 Lady Street, Columbia, SC, 29201, US

(21) Appl. No.: 09/736,900

(22) Filed: Dec. 13, 2000

Related U.S. Application Data
(60) Provisional application No. 60/170,922, filed on Dec. 15, 1999.

(51) Int. Cl. C12Q 1/02

(52) U.S. Cl. 435/29

(57) ABSTRACT

The present invention is directed to a method and device for measuring water quality. The present invention utilizes displaceable particles located upon a substrate for measuring the water quality. The device allows the particles to be displaced by attaching bacteria to the substrate. The presence or the amount of bacteria in water is detected over a relatively short time period so as to provide a device or method for near instantaneous indication or measurement of water quality.

The present invention is based on provisional patent application Serial No. 60/170,922 filed Dec. 15, 1999, and priority is hereby claimed therefrom.

### FIELD OF THE INVENTION

The present invention relates to a device and method for determining fluid quality. More specifically, the present invention relates to a device and method that provide a near instantaneous measurement or indication of the presence or concentration of bacteria in fluids such as water.

### BACKGROUND OF THE INVENTION

Bacterial diseases are frequently transmitted to humans through the ingestion or use of contaminated water. The spread of these diseases may be mitigated if the bacterial contamination is detected while present in small numbers and before the water is used for drinking, cleaning, recreation, and other contact activities. Detection is generally accomplished through routine microbiological examination and monitoring of samples taken at or near the point of use. In addition, appropriate monitoring is required after detection to determine the effectiveness of any treatment. Accordingly, water monitoring requirements exist worldwide to ensure public health.

The majority of the existing tests rely on the detection of total heterotrophic bacteria and other indicator organisms such as Escherichia coli. The presence of Escherichia coli, one of several coliform bacteria, indicates that the water has been contaminated with human feces and that the potential for pathogenic, disease-causing bacteria exists. Coliform bacteria are present in human intestinal wastes in substantial numbers and survive in water at least as well as the associated pathogens. Thus, the detection of coliform bacteria is a valid indicator for determining water quality.

Quality standards for drinking water, well water, bathing water, and open waters in the United States are established and enforced by the Environmental Protection Agency (EPA). EPA regulations specify the minimum frequency of water sampling and the maximum number of coliform organisms allowed. For example, the EPA has suggested that the bacterial counts in drinking water should not exceed 500 colony forming units per milliliter of water. The available and EPA-approved tests for detection and identification of total heterotrophic bacteria, total coliform bacteria, and fecal coliforms depend on the isolation and growth of bacteria on selective media. However, currently available testing techniques have well-known limitations, as they are labor intensive, require considerable time to confirm possible bacterial contamination and, in the case of tests based upon utilization of a polymerase chain reaction (PCR), are unable to distinguish viable bacteria from dead bacteria.

For example, the multiple-tube fermentation method is an EPA approved method that can be used to determine total count, concentration of coliforms, and the presence of fecal coliforms. The procedures for this method are cumbersome and require numerous expendables. The multiple-tube fermentation method for fecal coliforms is performed in three steps: a) the presumptive test; b) the confirmatory test; and c) the completed test. Furthermore, the method requires a high degree of operator training and is very time consuming, requiring considerable time and in some instances as much as three days to complete.

Bacterial concentrations in water can also be determined by growth on agar plates using techniques such as pour plate, spread plate, or membrane filtration. The membrane filter method is the most widely used of all methods for the testing of water. This procedure allows for measurements of total heterotrophs, coliforms, and fecal coliforms with the substitution of the selective and/or differential growth media. In this test, a water sample is drawn through a membrane filter so as to retain the bacteria on the surface of the membrane. The membrane is then placed on an appropriate agar medium or pad saturated with the medium and incubated. Colonies are counted and reported as the number of colony forming units (CFU) per volume of sample. The results of this test can be determined in approximately 48 hours.

Rapid water quality tests based on polymerase chain reaction (PCR) amplification of DNA are also able to detect bacteria at concentrations as low as a single bacterial cell per 100 ml of water. Unfortunately, PCR procedures require extreme control of the reaction conditions and processing steps to ensure the elimination of false positives. The use of PCR to quantify bacteria concentrations is also not practical since the reaction plateaus at around 10⁸ copies per amplicon. Furthermore, PCR requires confirmatory analysis of the DNA products, usually by either gel electrophoresis (incorporating staining, probe hybridization, capture tags), dot blots (probe hybridization), endonuclease digestion analysis (gel electrophoresis), high-pressure liquid chromatography (UV detection), electrochemiluminescence, scintillation proximity assay, or direct sequencing. Finally, PCR will give positive test results from the DNA or RNA of both live and dead target bacteria. Thus, the determination of the successfulness of water treatment steps taken after detection remains very impractical with this method.

Space flight creates additional, unique complications for water monitoring. With current closed loop water distribution systems, quick appraisals are even more critical since there is little time between water processing, testing, and consumption. In weightless or microgravity environments, the handling of liquids and test tubes during the testing procedures becomes impossible or impractical.

Therefore, a need exists for a device or method that measures or indicates the quality of fluids, such as the quality of water. More particularly, a need exists for a device or method that can indicate the presence of bacteria or measure the concentration of bacteria in a fluid such as water shortly after the testing begins. Additionally, a need exists for a device or method that may measure or indicate fluid quality in weightless or microgravity environments.

### SUMMARY OF THE INVENTION

The present invention is directed to a method and device for measuring the quality of fluids such as water. While the present invention is discussed in terms of determining the quality of drinking water, the present invention could be used to determine the quality of any fluid. For example, the quality of fluids such as wine, industrial effluents, and the like may be measured using the present invention.

More specifically, the present invention provides a method and device for determining or measuring the presence of bacteria in water in near real-time. In certain embodiments, the present invention provides a qualitative indicator for detecting whether bacteria are present in water. In other embodiments, the present invention provides a quantitative device for determining the amount or concentration of bacteria present in water.

The present invention utilizes displaceable particles located upon a substrate. Upon placing the substrate into the water being tested, bacteria present in the water displace the particles located upon the substrate. The particles may then be collected and measured to allow a determination of the amount or concentration of bacteria present in the water sample. Alternatively, the substrate and displaceable particles may be configured such that a visual effect takes place as the particles are displaced so as to provide an indication of the presence of bacteria in the water.

A method for determining water quality includes locating displaceable particles upon the surface of a substrate. The substrate is then submersed in the water. The water may be a sample of the water to be tested, or may actually be the source being tested such as a lake, river, container, faucet, or the like. While present in the water, there occurs a displacing of particles from the surface of the substrate with bacteria that are present in the water. The displaced particles may then be collected and measured to determine the water quality.

Similarly, a device for measuring water quality, in one particular embodiment, includes a substrate that may be submersed into water and that has a surface upon which displaceable particles are located. A collector is included for collecting the particles displaced by the bacteria after submersing the substrate into the water. Upon collecting the particles, a measuring unit may be used to measure the amount of particles displaced, which is then correlated to the amount of bacteria present in the water being tested.

Unlike existing tests, the present invention provides an indication or measurement of water quality in near real-time. More specifically, the present invention can provide an indication or measurement of water quality in a relatively short period of time after placing the substrate into the water being tested. Furthermore, the present invention is not sensitive to dead bacteria that may be present in the water being tested, and therefore may provide a more accurate determination of water quality than existing devices. The present invention does not require gravity or extensive procedures to operate and therefore is suitable for use in zero or microgravity environments.

Numerous embodiments of the present invention may be envisioned using the teachings disclosed herein. For example, the present invention may include embodiments for use with consumer water filters that are structured into existing water filtration devices so as to provide consumers with an indication of water quality or filter performance. In addition, embodiments for on-site testing of water in lakes, rivers, municipal water supplies, and the like may be structured. Furthermore, the present invention may include an embodiment for repetitive testing in a laboratory or water treatment facility

These and other features, aspects, and advantages of the present invention will become better understood with reference to the following description and appended claims.

### DETAILED DESCRIPTION OF REPRESENTATIVE EMBODIMENTS

Reference now will be made in detail to some of the embodiments of the invention, one or more examples of which are set forth below. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment, can be used on another embodiment to yield a still further embodiment. Thus, it is intended that the present invention cover such modifications and variations as come within the scope of the appended claims and their equivalents. Other objects, features and aspects of the present invention are disclosed in or are obvious from the following detailed description. It is to be understood by one of ordinary skill in the art that the present discussion is a description of exemplary embodiments only, and is not intended as limiting the broader aspects of the present invention.

As used herein, the term [symbol] displaceable particles [symbol] means particles that remain bound to a substrate when the substrate is submersed into bacteria free water under normal flow either past or through the surface of the substrate, or when the substrate is otherwise subjected to mechanical manipulation. However, the particles are subject to displacement by bacteria from the surface of the substrate when placed into water containing bacteria. In the event no bacteria are present in the water, the particles are still displaceable but are not actually displaced. The force that binds the particle to the substrate may be an electrostatic force, modifiable chemical force, disintegratable mechanical bonds, or van der Waals type forces.

As used herein, the term [symbol] charge-modified material [symbol] means any material that has an electric charge upon at least some of its surfaces. The charge may be cationic or anionic, and of any magnitude.

As used herein, the term “nonwoven web” means a web or fabric having a structure of individual fibers or threads which are interlaid, but not in an identifiable manner as in a knitted or woven fabric. Nonwoven webs generally may be prepared by methods which are well known to those having ordinary skill in the art. Examples of such processes include, by way of illustration only, meltblowing, coforming, spunbonding, carding and bonding, air laying, and wet laying. Meltblowing, coforming, and spunbonding processes are exemplified by the following references, each of which is incorporated herein by reference:

(a) meltblowing references include, by way of example, U.S. Pat. No. 3,016,599 to R. W. Perry, Jr., U.S. Pat. No. 3,704,198 to J. S. Prentice, U.S. Pat. No. 3,755,527 to J. P. Keller et al., U.S. Pat. No. 3,849,241 to R. R. Butin et al., U.S. Pat. No. 3,978,185 to R. R. Butin et al., and U.S. Pat. No. 4,663,220 to T. J. Wisneski et al. See, also, V. A. Wente, “Superfine Thermoplastic Fibers”, Industrial and Engineering Chemistry, Vol. 48, No. 8, pp.1342-1346 (1956); V. A. Wente et al., “Manufacture of Superfine Organic Fibers”, Navy Research Laboratory, Washington, D.C., NRL Report 4364 (111437), dated May 25, 1954, United States Department of Commerce, Office of Technical Services; and Robert R. Butin and Dwight T. Lohkamp, “Melt Blowing—A One-Step Web Process for New Nonwoven Products”, Journal of the Technical Association of the Pulp and Paper Industry, Vol. 56, No.4, pp. 74-77 (1973);

(b) coforming references include U.S. Pat. No. 4,100,324 to R. A. Anderson et al. and U.S. Pat. No. 4,118,531 to E. R. Hauser; and

(c) spunbonding references include, among others, U.S. Pat. No. 3,341,394 to Kinney, U.S. Pat. No. 3,655,862 to Dorschner et al., U.S. Pat. No. 3,692,618 to Dorschner et al., U.S. Pat. No. 3,705,068 to Dobo et al., U.S. Pat. No. 3,802,817 to Matsuki et al., U.S. Pat. No. 3,853,651 to Porte, U.S. Pat. No. 4,064,605 to Akiyama et al., U.S. Pat. No. 4,091,140 to Harmon, U.S. Pat. No. 4,100,319 to Schwartz, U.S. Pat. No. 4,340,563 to Appel and Morman, U.S. Pat. No. 4,405,297 to Appel and Morman, U.S. Pat. No. 4,434,204 to Hartman et al., U.S. Pat. No. 4,627,811 to Greiser and Wagner, and U.S. Pat. No. 4,644,045 to Fowells.

A [symbol] nonwoven charge-modified microfiber glass web [symbol] may be prepared from a fibrous web which incorporates glass fibers having a cationically charged coating thereon. Generally, such microfibers would be glass fibers having an average diameter of about 10 microns or less. The coating includes a functionalized cationic polymer which has been crosslinked by heat; in other words, the functionalized cationic polymer has been crosslinked by heat after being coated onto the glass fibers. Such fibrous filter is prepared by a method which involves providing a fibrous filter which includes glass fibers, passing a solution of a functionalized cationic polymer crosslinkable by heat through the fibrous filter under conditions sufficient to substantially coat the fibers with the functionalized cationic polymer, and treating the resulting coated fibrous filter with heat at a temperature and for a time sufficient to crosslink the functionalized cationic polymer present on the glass fibers. The functionalized cationic polymer may be an epichlorohydrin-functionalized polyamine or an epichlorohydrin-functionalized polyamido-amine.

In general, when used as a filter media, a [symbol] charge-modified microfiber glass web [symbol] will contain at least about 50 percent by weight of glass fibers, based on the weight of all fibers present in the filter media. In some embodiments, essentially 100 percent of the fibers will be glass fibers. When other fibers are present, however, they generally will be cellulosic fibers, fibers prepared from synthetic thermoplastic polymers, or mixtures thereof.

As used herein, the terms [symbol] cationically charged [symbol] in reference to a coating on a glass fiber and [symbol] cationic [symbol] in reference to the functionalized polymer mean the presence in the respective coating and polymer of a plurality of positively charged groups. Thus, the terms [symbol] cationically charged [symbol] and [symbol] positively charged [symbol] are synonymous. Such positively charged groups typically will include a plurality of quaternary ammonium groups, but they are not necessarily limited thereto.

The term [symbol] functionalized [symbol] is used herein to mean the presence in the cationic polymer of a plurality of functional groups, other than the cationic groups, which are capable of crosslinking when subjected to heat. Thus, the functional groups are thermally crosslinkable groups. Examples of such functional groups include epoxy, ethylenimino, and episulfido. These functional groups readily react with other groups typically present in the cationic polymer. The other groups typically have at least one reactive hydrogen atom and are exemplified by amino, hydroxy, and thiol groups. It may be noted that the reaction of a functional group with another group often generates still other groups which are capable of reacting with functional groups. For example, the reaction of an epoxy group with an amino group results in the formation of a β-hydroxyamino group.

Thus, the term [symbol] functionalized cationic polymer [symbol] is meant to include any polymer which contains a plurality of positively charged groups and a plurality of other functional groups which are capable of being crosslinked by the application of heat. Particularly useful examples of such polymers are epichlorohydrin-functionalized polyamines and epichlorohydrin-functionalized polyamido-amines. Both types of polymers are exemplified by the Kymene [symbol] resins which are available from Hercules Inc., Wilmington, Del. Other suitable materials include cationically modified starches, such as CoBond, from National Starch.

As used herein, the term [symbol] thermally crosslinked [symbol] means the coating of the functionalized cationic polymer has been heated at a temperature and for a time sufficient to crosslink the above-noted functional groups. Heating temperatures typically may vary from about 50 [symbol] C to about 150 [symbol] C. Heating times in general are a function of temperature and the type of functional groups present in the cationic polymer. For example, heating times may vary from less than a minute to about 60 minutes or more.

As discussed briefly above, a nonwoven charge-modified meltblown web may consist of hydrophobic polymer fibers, amphiphilic macromolecules adsorbed onto at least a portion of the surfaces of the hydrophobic polymer fibers, and a crosslinkable, functionalized cationic polymer associated with at least a portion of the amphiphilic macromolecules, in which the functionalized cationic polymer has been crosslinked. Crosslinking may be achieved through the use of a chemical crosslinking agent or by the application of heat. Desirably, thermal crosslinking, i.e., the application of heat, will be employed. In general, the amphiphilic macromolecules may be of one or more of the following types: proteins, poly(vinyl alcohol), monosaccharides, disaccharides, polysaccharides, polyhydroxy compounds, polyamines, polylactones, and the like. Desirably, the amphiphilic macromolecules will be amphiphilic protein macromolecules, such as globular protein or random coil protein macromolecules. For example, the amphiphilic protein macromolecules may be milk protein macromolecules. The functionalized cationic polymer typically may be any polymer which contains a plurality of positively charged groups and a plurality of other functional groups which are capable of being crosslinked by, for example, chemical crosslinking agents or the application of heat. Particularly useful examples of such polymers are epichlorohydrin-functionalized polyamines and epichlorohydrin-functionalized polyamido-amines. Other suitable materials include cationically modified starches.

The nonwoven charge-modified meltblown web may be prepared by a method which involves providing a fibrous meltblown filter media which includes hydrophobic polymer fibers, passing a solution containing amphiphilic macromolecules through the fibrous filter under shear stress conditions so that at least a portion of the amphiphilic macromolecules are adsorbed onto at least some of the hydrophobic polymer fibers to give an amphiphilic macromolecule-coated fibrous web, passing a solution of a crosslinkable, functionalized cationic polymer through the amphiphilic macromolecule-coated fibrous web under conditions sufficient to incorporate the functionalized cationic polymer onto at least a portion of the amphiphilic macromolecules to give a functionalized cationic polymer-coated fibrous web in which the functionalized cationic polymer is associated with at least a portion of the amphiphilic macromolecules, and treating the resulting coated fibrous filter with a chemical crosslinking agent or heat. Desirably, the coated fibrous filter will be treated with heat at a temperature and for a time sufficient to crosslink the functionalized cationic polymer.

In general, the present invention relates to a method and device for measuring water quality. More specifically, the present invention provides a method and device for determining in a relatively short time period whether bacteria are present in the water being tested. The present invention includes embodiments that may provide a qualitative indicator for detecting whether bacteria are present in water without determining the amount or concentration of bacteria present. In other embodiments, the present invention provides a quantitative device for determining the amount or concentration of bacteria present in the water being tested.

A method of determining water quality may include locating displaceable particles upon the surface of a substrate, and then submersing the substrate in water. After displacing a portion of the particles with bacteria present in the water, the displaced particles are collected. The collected particles are then measured to determine the water quality.

A device for measuring water quality, in one embodiment, may include a substrate, for submersing in water. Upon the surface of the substrate are located displaceable particles. A collector is provided for collecting the particles displaced by bacteria after the substrate is submersed into water. A measuring unit is included so that the collected particles displaced from the surface may be measured and the water quality thereby determined.

Locating the displaceable particles upon the surface of a substrate may be accomplished by any process that places the particles and the surface into contact with each other. By way of example, the substrate may be immersed into a bed of the particles, the particles may be poured onto the surface, the particles may be sprayed on the surface, and the like. The particles should be located upon the surface such that the particles are displaceable by bacteria present in the water.

The particles and substrate are specifically selected from materials that will facilitate the displacement of the particles from the surface of the substrate. While the present invention is not to be limited by any particular theory the inventors may have as to a plausible explanation for the mechanism by which the invention operates, it is theorized that the present invention utilizes competitive displacement of the particles by the bacteria. Specifically, the inventors believe that the particles are attached to the substrate through a binding force. Accordingly, the particles and substrate are selected such that a binding force exists between both the substrate and the particles, and the substrate and the bacteria. It is believed that the binding force between the particles and the substrate must be less than the binding force between the bacteria and the substrate. Under these conditions, the bacteria coming into the proximity of the substrate [symbol] s surface displace the particles and become located upon the surface.

The particles may be microscopic in size and exhibit paramagnetic properties. For example, the particles may be microparticles having an iron core. The particles may also be configured to have a precise charge to facilitate the displacement and collection of the particles.

The substrate should be selected such that an attractive force exists between the particles and the substrate. However, it is believed that this attractive force should not exceed the attractive force between the bacteria and the substrate. Preferably, the substrate will be constructed from a charge-modified media. The substrate should also be compatible for contact with water and, in some cases, prolonged contact with water. By way of example only, the substrate may be constructed from a charge-modified meltblown web, a charge-modified microfiber glass web, and other charge-modified materials. Additionally, the specific particle/substrate combination may be selected so that the particles are displaced by only certain types of bacteria. In this way, the present invention may be used to test water for specific bacterial contaminants.

After the displaceable particles have been located upon the surface of the substrate, the substrate is submersed into the water being tested. The water may be a sample, or may be the actual water source being tested. For example, the substrate may be submersed into water from a municipal water supply, sink, river, lake, well, container, test tube, or the like. Preferably, the substrate is submersed into water that may flow across or through the surface of the substrate so that bacteria may be brought into the proximity of the particles thereby allowing displacement of the particles from the surface to occur. Accordingly, the substrate may be placed into a flowing stream or the substrate is placed into motion such that water is passing across or through the surface of the substrate. The substrate remains submersed in water containing bacteria for a time period sufficient to allow displacing of a portion of the particles from the surface of the substrate by the bacteria present in the water source. This time period is relatively short and allows the present invention to provide a near instantaneous measurement of water quality.

After displacement of the particles from the surface of the substrate, the particles may be collected. Embodiments of the present invention may include a collector for collecting all of the displaced particles. Preferably, the collector uses a magnetic field to attract and hold paramagnetic microparticles. For example, a magnet or an electromagnetic coil may be used to collect microparticles that have an iron core and are displaceable by the bacteria. The collector may also be structured to permit recovery and reuse of the particles. Accordingly, the present invention may include a means for removing the bacteria from the surface of the substrate or for replacing the substrate, and then relocating the collected particles upon the surface of the substrate.

Following collection of the particles, the particles may be measured to determine the amount of bacteria present in the water sample or the concentration of bacteria in the water. Embodiments of the present invention may include a measuring unit for measuring the displaced particles. For example, the measuring unit may be scales by which the collected particles are weighed. The scales may be directly connected to the collector for obtaining the total weight of the particles collected. Knowing the weights of the individual particles and the displacement ratio of bacteria to particles (e.g. 1 to 1, 2 to 1, and the like), the number of bacteria may be determined. Using this number and the volume of the water sample being tested, the concentration of bacteria in the sample may be readily calculated. For applications where the measuring unit is placed into a moving stream of water, the concentration of bacteria in the water stream may be calculated by determining the number of displaced particles, the volumetric flow rate of the water stream, and the time during which the substrate was submersed into the water stream.

In addition, embodiments of the invention may include a measuring unit for determining the number of particles that remain upon the surface of the substrate as opposed to determining the number of particles displaced. For example, the substrate may be weighed before and after displacement to determine the number of particles remaining upon the surface of the substrate.

Alternatively, the measuring unit and collector may be constructed from a piezoelectric crystal attached to a magnet. As particles are collected by the magnet, a charge build-up on the piezocrystal occurs than can be measured and correlated with the number of particles. As above, determining the number of displaced particles allows determination of the number or concentration of bacteria present in the water tested. The piezocrystal could be used in conjunction with an alarm system so as to send an audio or visual warning when bacteria are detected. Various other measuring units and embodiments of the present invention may be envisioned using the teachings disclosed herein.

In some embodiments, the present invention may simply provide a qualitative measurement of water quality. More specifically, the present invention may simply be used to indicate whether bacteria are present in the water. For example, the present invention may by structured such that a visual change takes place as the bacteria displace the particles. In one embodiment, the substrate and particles may be of the same color. As the bacteria displace the particles, a color change occurs that may be visually detected. Similarly, the substrate could be shaped into letters such that a word appears as displacement occurs.

For example, an embodiment could be structured with a surface of charge-modified media forming the word [symbol] CONTAMINATED. [symbol] As particles having the same color as the surface of the charge-modified substrate are displaced, the word [symbol] CONTAMINATED [symbol] would be made to appear. Alternatively, the particles may be selected as a different color than the media such that an image fades from view as displacement occurs.

The present invention provides a near instantaneous determination of water quality by providing a measurement or indication of bacteria present in water. Accordingly, embodiments of the present invention may be installed into the plumbing of a residence or office for monitoring water quality. Embodiments for providing an indication or measurement of bacteria could be located at the point-of-entry for the water supply to a business or dwelling. Alternatively, such embodiments could be placed at various points-of-use throughout the business or dwelling such as faucets, showers, ice-makers, and the like.

The present invention may be structured for use with various water filtration devices. For example, an embodiment of the present invention may be located upstream of a filter to provide an instantaneous or cumulative indicator of bacteria present in the water being filtered. Alternatively, an embodiment may be located downstream of the filter to provide an indication of whether a filter is adequately removing bacteria from the water supply or to notify a consumer to replace the filter media. The substrate may be structured to allow flow across the surface, or could be located within the filter media and constructed of porous material such that water flows through the substrate and the filter media.

Small, disposable embodiments of the present invention may be used to allow for rapid field testing of municipal water supplies for bacterial contamination. For example, an embodiment may include the substrate located upon a paddle or test rod. The user would stir the paddle or rod in the water being tested while looking for a visual change to indicate whether bacterial contaminants are present. Additionally, potable embodiments of the present invention may be constructed for circulating the water through or across the substrate at a known volumetric flow rate for prescribed time periods. By determining the number of displaced particles, the concentration of bacteria may be determined using a device that is portable, nearly instantaneous, and does not require extensive equipment.

Additionally, the present invention does not require a gravity environment to operate. Regardless of the presence of gravity, the flow of water through or across the substrate can be created through equipment such as a pump or centrifuge. Additionally, embodiments of the invention may be structured for use in the closed systems required for space flight and microgravity environments to provide a rapid measurement or indication of water quality. Current systems used in space flight may recycle human wastes such as urine for use as drinking water. The present invention provides a device and method by which bacteria may be detected so that preventative action and monitoring may be undertaken in the event of contamination. In addition, embodiments of the present invention may be configured to occupy a minimum of space and weight—a requirement for space flight.

Although preferred embodiments of the invention have been described using specific terms, devices, and methods, such description is for illustrative purposes only. The words used are words of description rather than of limitation. It is to be understood that changes and variations may be made by those of ordinary skill in the art without departing from the spirit or the scope of the present invention, which is set forth in the following claims. In addition, it should be understood that aspects of the various embodiments may be interchanged both in whole or in part. Therefore, the spirit and scope of the appended claims should not be limited to the description of the preferred versions contained therein.

What is claimed is:

1. A method of measuring water quality, comprising:
locating displaceable particles upon the surface of a substrate;
submersing the substrate in water;
displacing a portion of the particles from the surface of the substrate with any bacteria present in the water;
collecting any displaced particles; and
measuring the amount of displaced particles to determine the water quality.

2. A method as in claim 1, wherein the substrate is a charge-modified media.

3. A method as in claim 1, wherein the particles comprise iron.

4. A method as in claim 1, wherein the particles are paramagnetic microparticles.

5. A method as in claim 4, wherein the collecting of paramagnetic microparticles is accomplished using a magnetic field.

6. A method as in claim 1, wherein the collecting of particles is accomplished by using a magnet.

7. A method as in claim 6, wherein measuring the amount of displaced particles is accomplished by connecting a piezoelectric crystal to the magnet such that a measurable electrical charge is created as the particles are collected by the magnet.

8. A method as in claim 1, wherein measuring the amount of displaced particles is performed by weighing the displaced particles.

9. A method as in claim 1, further comprising passing water over the surface of the substrate after submersing the substrate in the water.

10. A method as in claim 1, further comprising regenerating the substrate by returning the collected particles to the substrate after measuring the amount of particles.

11. A method of determining water quality, comprising:
locating displaceable particles upon the surface of a substrate so as to obscure an image present on the surface of the substrate submersing the substrate in water; and
displacing particles from the surface of the substrate with any bacteria present in the water such that a visual change upon the surface of the substrate occurs if there is bacteria present in the water.

12. A method as in claim 11, wherein the particles and the substrate are of the same color.

13. A method as in claim 11, wherein the substrate is a charge-modified media.

14. A method as in claim 11, wherein the particles comprise iron.

15. A method as in claim 11, wherein the particles are paramagnetic microparticles.

16. A method as in claim 11, further comprising passing water over the surface of the substrate after submersing the substrate in the water.

17. A method as in claim 11, further comprising regenerating the substrate by returning the particles to the substrate after the visual change occurs upon the surface of the substrate.

18. A device for measuring water quality, comprising:
a substrate, for submersing in water;
displaceable particles located upon the surface of the substrate
a collector for collecting any particles displaced by bacteria after submersing the substrate into the water; and
a measuring unit for measuring the amount of any particles displaced so that the water quality may be determined.

19. A device as in claim 18, wherein the substrate comprises a charge-modified media.

20. A device as in claim 18, wherein the particles comprise iron.

21. A device as in claim 18, wherein the particles comprise paramagnetic microparticles.

22. A device as in claim 18, wherein the collector comprises a magnetic field.

23. A device as in claim 18, wherein the collector comprises a magnet.

24. A device as in claim 23, further comprising a piezoelectric crystal connected to the magnet such that displaced particles collected by the magnet cause an electrical charge that may be measured.

25. A device as in claim 18, wherein the measuring unit is an apparatus for determining the weight of the displaced particles.

26. A device for indicating water quality, comprising:
a substrate, for submersing in water; and
displaceable particles located upon the surface of the substrate;
said particles being constructed such that upon submersing the substrate in the water the particles are displaced by any bacteria present in the water to effect a visual change upon the surface of the substrate if there is bacteria present in the water.

27. A device for indicating water quality as in claim 26, wherein the substrate comprises a charge-modified media.

28. A device as in claim 26, wherein the particles comprise iron.

29. A device as in claim 26, wherein the particles comprise paramagnetic microparticles.

30. A device as in claim 26, wherein the particles and the substrate are of the same color.

31. A device as in claim 26, wherein the particles and the substrate are of different colors.

32. A method of determining water quality, comprising:
locating displaceable particles upon the surface of a substrate so as to create an image on the surface of the substrate
submersing the substrate in water; and
displacing particles from the surface of the substrate with any bacteria present in the water such that a visual change in the image occurs if there is bacteria present in the water

33. A method as in claim 32, wherein the particles and the substrate are of different colors.

34. A method as in claim 32, wherein the substrate is a charge-modified media.

35. A method as in claim 32, wherein the particles comprise iron.

36. A method as in claim 32, wherein the particles are paramagnetic microparticles.

37. A method as in claim 32, further comprising passing water over the surface of the substrate after submersing the substrate in the water.

38. A method as in claim 32, further comprising regenerating the substrate by returning the particles to the substrate after the visual change occurs upon the surface of the substrate.

39. A method for detecting bacteria in a fluid comprising the steps of:
providing a substrate adapted to attract bacteria having a first attractive force, the substrate having particles bound to a surface thereof by a second attractive force which is less than the first attractive force;
submersing the substrate in the fluid to allow any bacteria present in the fluid to displace the particles; and
detecting whether any particles were displaced from the surface of the substrate by bacteria.

40. A method as in claim 39, wherein the particles and the substrate are of different colors.

41. A method as in claim 39, wherein the substrate is a charge-modified media.

42. A method as in claim 39, wherein the particles comprise iron.

43. A method as in claim 39, wherein the particles are paramagnetic microparticles.

44. A method as in claim 39, further comprising passing the fluid over the surface of the substrate after submersing the substrate in the fluid.

45. A method as in claim 39, further comprising regenerating the substrate by returning the particles to the substrate after detecting whether any particles were displaced from the surface of the substrate by bacteria.
